(19) United States
(12) Patent Application Publication
(10) Pub. No.: US 2025/0177192 A1
(43) Pub. Date: Jun. 5, 2025

(54) APPARATUS FOR INVISIBLE TOOTH ALIGNMENT WITH ORTHO STRUT FOR SLEEP APNEA

(71) Applicant: ACHAEMENID, LLC, Boston, MA (US)

(72) Inventor: Reza RADMAND, Boston, MA (US)

(21) Appl. No.: 19/042,968

(22) Filed: Jan. 31, 2025

Related U.S. Application Data
(63) Continuation-in-part of application No. 17/321,597, filed on May 17, 2021, now Pat. No. 12,048,646.
Application No. 17/321,597 is a continuation of application No. 16/871,477, filed on May 11, 2020, now Pat. No. 12,213,907.
(60) Provisional application No. 63/627,771, filed on Jan. 31, 2024.
(60) Provisional application No. 62/889,383, filed on Aug. 20, 2019.

(30) Foreign Application Priority Data
Nov. 9, 2018 (WO) PCT/US2018/060066

(51) Int. Cl. A61F 5/56

(52) U.S. Cl. CPC A61F 5/566

(57) ABSTRACT

An oral aligning appliance uses upper and lower aligning trays to align a user's dentition while treating sleep apnea. The upper and lower aligning trays may be connected to each other by a strut removably connected to each of the upper and lower alignment trays and configured for moving the user's lower jaw forward for treating sleep apnea while the upper and lower alignment trays align the user's dentition. One or more of the upper and lower alignment trays and strut may be periodically replaced as part of progressive alignment treatment or adjustments to the sleep apnea therapy carried out at the same time.

### CROSS REFERENCE TO RELATED APPLICATIONS

This application claims priority to U.S. Provisional Patent Application No. 63/627,771, filed on Jan. 31, 2024, the entire content of each of which is being incorporated by reference herein, and is a Continuation in Part of U.S. application Ser. No. 17/321,597 filed May 17, 2021, which is a Continuation Application of U.S. application Ser. No. 16/871,477 filed May 11, 2020, which is a Continuation-in-Part of International Application No. PCT/US2018/060066 filed Nov. 9, 2018, which claims the benefit of U.S. Provisional Patent Application No. 62/585,145, filed Nov. 13, 2017, U.S. Provisional Patent Application No. 62/592,857, filed Nov. 30, 2017, U.S. Provisional Patent Application No. 62/595,712, filed Dec. 7, 2017, U.S. Provisional Patent Application No. 62/678,287, filed May 31, 2018, U.S. Provisional Patent Application No. 62/678,292, filed May 31, 2018, and U.S. Provisional Patent Application No. 62/689,380, filed Jun. 25, 2018, each of which is incorporated herein by reference in its entirety. U.S. application Ser. No. 16/871,477 claims the benefit of U.S. Provisional Patent Application No. 62/889,383 filed Aug. 20, 2019, which is incorporated herein by reference in its entirety.

### FIELD OF THE DISCLOSURE

An oral sleep appliance including an orthodontic aligner and associated method for providing both treatment of obstructive sleep apnea and correcting misaligned dentition in a user.

### BACKGROUND OF THE DISCLOSURE

Sleep apnea is a common medical condition during which a person experiences one or more pauses in breathing and/or shallow breaths during sleep. While there are several types of sleep apnea, the most common type is obstructive sleep apnea. In this medical condition, one or more of the person's throat muscles relax during sleep causing surrounding tissues in the posterior portions of the mouth, nose, and throat to collapse and block the airway. Persons suffering from obstructive sleep apnea have inadequate oxygen exchange during sleep, which can lead to daytime fatigue, lack of concentration, and mood changes. Left untreated, obstructive sleep apnea can have a significant impact on a person's health, often leading to cardiovascular, stroke, and metabolic disorders.

Known methods for treatment of obstructive sleep apnea include both surgical methods or interventions and nonsurgical devices. A popular surgical procedure is uvulopalatopharyngoplasty, whereby a portion of the soft palate is removed in an effort to prevent closure of the airway by excess tissue during sleep. A disadvantage of this procedure, however, is that the operation is often expensive and may damage throat muscles necessary for swallowing and/or cause other undesirable disorders, such as nasal regurgitation. To reduce this risk, various nonsurgical approaches have been employed. Once such nonsurgical approach includes using standardized custom oral sleep appliances to incrementally advance and/or protrude the mandible (lower jaw) relative to the maxilla (upper jaw). These standardized appliances, commonly referred to as mandibular advancement devices (“MADs”), typically include upper and lower dental trays, in which the lower dental tray is designed to advance the mandible, and hence, move the tongue forward to increase the space in the posterior part of the throat and the oropharynx, which in turn may serve to increase the flow of air during sleep. The distance (degree of advancement) required to protrude and/or reposition the mandible may be, at least in part, dependent on the severity of the individual's obstructive sleep apnea, as well as psychological variables among the users.

Orthodontic therapy with the use of progressive aligners is known for correcting misaligned dentition and thereby, e.g., a patient's bite and smile line. MAD has a progressive mechanism to pull the lower jaw forward and allow for the tongue to move away from the back of the throat hence increase the volume of the air to exchange. One of the unintended consequences of MAD therapy is shifting of the teeth, especially upper and lower front teeth. A patient who has been successfully using the MAD may develop malformation of their dentition, forcing the patient to make a choice between discontinuing the treatment that in essence is saving their life from nightly episodes of oxygen deprivation or remaining on treatment with MAD while their dentition will continue to be misaligned and their bite progressively shifts away from what was once normal. In addition, using the MAD while sleeping may prevent a patient from beginning an alignment protocol because the aligners cannot be used during sleep.

Accordingly, devices and methods that facilitate concurrent use of MAD and progressive aligners would be beneficial.

### BRIEF DESCRIPTION OF THE EXEMPLARY EMBODIMENTS

In an aspect, this disclosure is directed to a strut for an oral aligning appliance. The strut may include a unitary body having an anterior end and a posterior end, a posterior projection connected to the posterior end, an anterior projection connected the anterior end. The anterior projection and the posterior projection may each have a shape of a spherical segment.

In another aspect, this disclosure is directed to an oral aligning appliance for concurrently treating sleep apnea and aligning dentition in a user. The oral aligning appliance may include an upper alignment tray configured for receiving upper teeth of the user and a lower alignment tray configured for receiving the lower teeth of the user. Each of the upper and lower alignment trays may have a first female housing positioned on a first side of the alignment tray and a second female housing positioned on an opposite side of the alignment tray. The oral aligning appliance may also include a pair of struts pivotably joined to each of the upper alignment tray and the lower alignment tray. Each strut may include a body, a posterior projection, and an anterior projection, wherein each of the posterior projections and the anterior projections is removably connected to a corresponding female housing and each of the anterior projections and the posterior projections have a shape of a spherical segment.

In another aspect, this disclosure is directed to a method for forming and/or using an oral aligning appliance according to this disclosure. The method includes making an impression of a user's dentition and fabricating a series of one or both of upper and lower alignment trays (upper and lower arch) for treating sleep apnea while progressively aligning the dentition of the user. The method further includes removably connecting a first strut to each of a first upper alignment tray and a first lower alignment tray, removing the first strut from one or both of the first upper alignment tray and the first lower alignment tray, and replacing at least one of the first strut, the first upper alignment tray, and the first lower alignment tray with a corresponding at least one of a second strut, a second upper alignment tray, and a second lower alignment tray. The method may further include reassembling the oral aligning appliance with the at least one of the second strut, second upper alignment tray, and second lower alignment tray, as part of the progressively aligning the dentition or treating sleep apnea.

### BRIEF DESCRIPTION OF THE DRAWINGS

A more particular description will be rendered by reference to specific embodiments thereof that are illustrated in the appended drawings. Understanding that these drawings depict only typical embodiments thereof and are not therefore to be considered to be limiting of its scope, exemplary embodiments will be described and explained with additional specificity and detail through the use of the accompanying drawings in which:

FIG. 1 is a perspective view of an exemplary oral appliance according to an embodiment;

FIG. 2 is a top view of the oral appliance of FIG. 1;

FIG. 3 is a side view of the oral appliance of FIG. 1;

FIG. 4 is a bottom view of an exemplary upper alignment tray according to an embodiment;

FIG. 5 is a bottom view of an exemplary lower alignment tray according to an embodiment;

FIG. 6 is a top, left perspective view of an exemplary strut according to an embodiment;

FIG. 7 is a front view of the strut of FIG. 6;

FIG. 8 is a partially exploded view of an exemplary oral appliance according to an embodiment;

FIG. 9 is a front view of an exemplary female housing according to an embodiment; and

FIG. 10 is a right perspective view of an oral appliance in an open position according to an embodiment.

Various features, aspects, and advantages of the embodiments will become more apparent from the following detailed description, along with the accompanying figures in which like numerals represent like components throughout the figures and text. The various described features are not necessarily drawn to scale but are drawn to emphasize specific features relevant to some embodiments.

The headings used herein are for organizational purposes only and are not meant to limit the scope of the description or the claims. To facilitate understanding, reference numerals have been used, where possible, to designate like elements common to the figures.

### DETAILED DESCRIPTION

For purposes of illustrating features of the embodiments, an example will now be introduced and referenced throughout the disclosure. Those skilled in the art will recognize that this example is illustrative and not limiting and is provided purely for explanatory purposes.

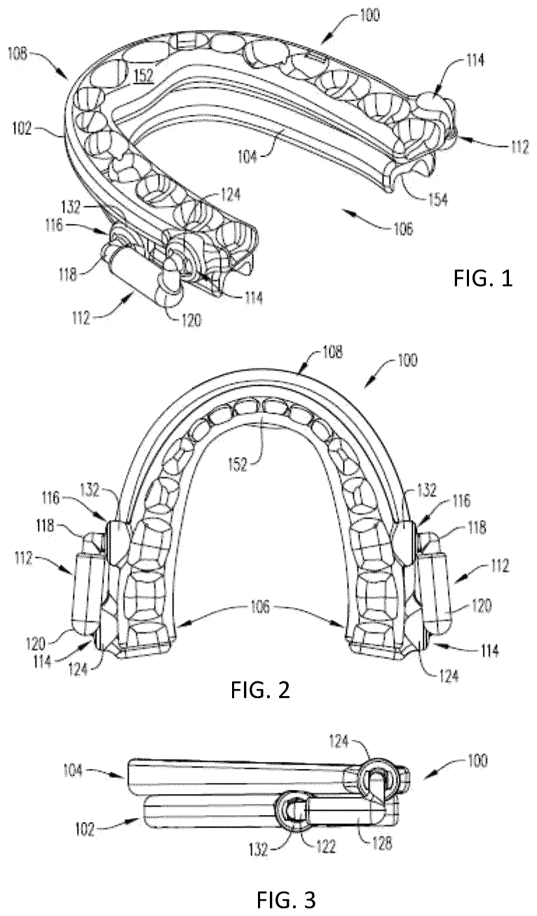

In one aspect, this disclosure is directed to an oral appliance for the temporary treatment of sleep apnea and contemporaneous progressive alignment of dentition. For example, FIGS. 1-3 illustrate an exemplary oral appliance (sometimes simply referred herein to as “oral appliance” or “appliance”) 100 generally including a first (i.e., upper) plate/tray 102 and a second (i.e., lower) plate/tray 104 in an opposed, facing relationship with one another. The upper tray 102 and lower tray 104 each include a respective posterior end (or portion) along a posterior end (or portion) 106 of the appliance 100 and an anterior end (or portion) along an anterior end (or portion) 108 of the appliance 100. The first tray 102 and the second tray 104 may be provided in various standard/generic sizes (similar to sports mouth guards), such as small, medium, large, or extra-large. The selected size may be based on the individual patient's jaw size, which helps to provide for a comfortable fit to the patient's dentition (i.e., configuration of teeth and gums). As will be understood by those of skill in the art, the appliance 100 is intended to be worn on the teeth of a user, such that the upper tray 102 receives the upper teeth of the user and the lower tray 104 receives the lower teeth of the user. In an aspect, the upper tray 102 and lower tray 104 may be conformed to the patient's teeth and/or designed for progressively aligning (i.e., shifting) the patient's teeth as is known.

The appliance 100 may be adjusted so that the lower tray 104 is offset from the upper tray 102 to maintain the user's lower jaw in a protruded (i.e., forwardly urged) position, in accordance with treatment protocol for sleep apnea. For example, the lower tray 104 may be urged forward (i.e., offset) a distance of 10 mm or more (e.g., in up to about 0.5 mm increments), for example, from about 2 mm to about 5 mm, for example, about 3.5 mm. Urging of the lower jaw in a forward direction helps to advance the patient's mandible and move the tongue forward, thereby increasing the space in the posterior part of the patient's throat and the oropharynx to increase the flow of air during the patient's sleep.

The exemplary appliance 100 includes a pair of struts or strut assemblies 112 pivotably (e.g., rotatably) connected to the upper tray 102 and lower tray 104 at respective upper (i.e., posterior) and lower (i.e., anterior) joints or hinges 114, 116, for example, ball-type joints (e.g., ball/socket joints) positioned along opposed lateral sides of the appliance 100. When the upper tray 102 and lower tray 104 are in a closed position according to the exemplary appliance 100 shown in FIGS. 1-3, the strut assemblies 112 extend generally along the outside of the lower tray 102 and turn upwards, extending to the upper tray 104.

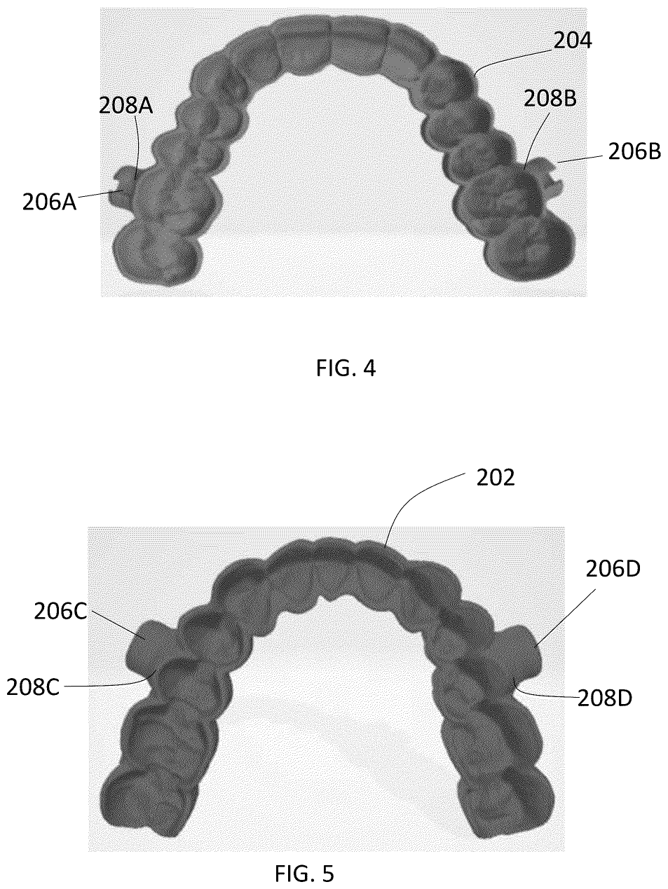

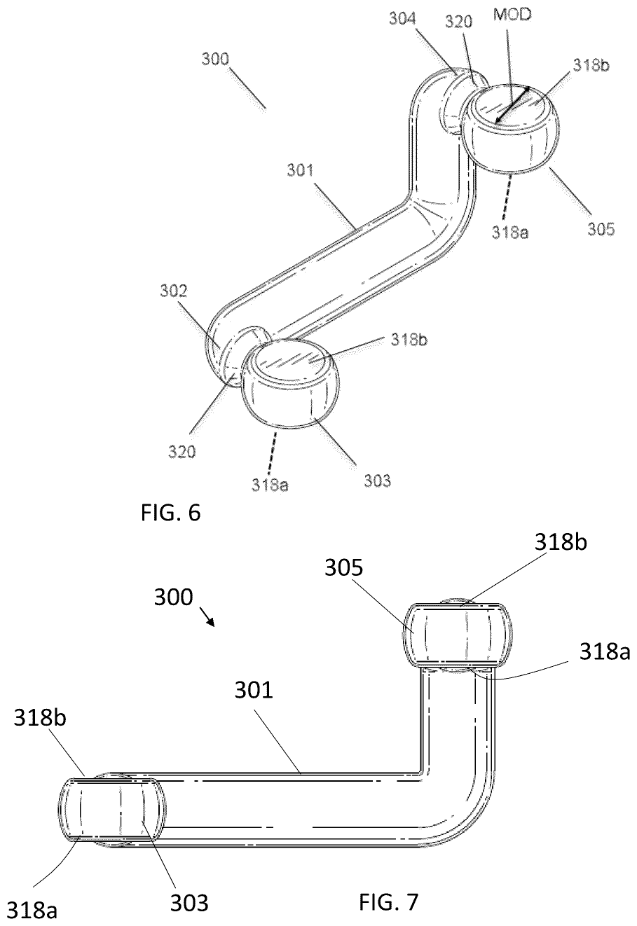

FIG. 4 and FIG. 5 respectively illustrate exemplary embodiments of an upper alignment tray 204 and a lower alignment tray 202 configured for use in an oral appliance according to this disclosure and as generally shown in FIGS. 1-3. For purposes of this disclosure, “alignment tray” refers to an orthodontic aligner as is known for progressive correction of misalignment, etc. In the exemplary embodiments shown in FIGS. 4 and 5, the upper alignment tray 204 and the lower alignment tray 202 may be configured for use with progressive detachable arms—i.e., struts 300 (FIGS. 6-8)—in succession. Each upper alignment tray and/or lower alignment tray, individually or as an upper/lower alignment tray pair, may be used for a recommended period of a number of days (typically 7-14 days) before changing the tray or trays to the next set, to correct pre-existing misaligned dentition or teeth that shift during the prescribed use of mandibular advancement device for the treatment of obstructive sleep apnea. For example, the upper alignment tray 204 and the lower alignment tray 202 in the exemplary embodiments each have a female housing 206 attached to either side.

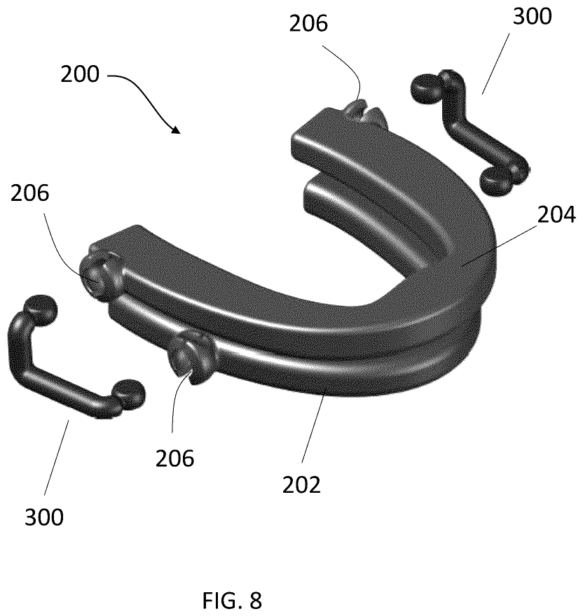

More specifically, the upper alignment tray 204 may include a female housing 206a with a base 208a and a female housing 206b with a base 208b and the lower alignment tray 202 may include a female housing 206c with a base 208c and a female housing 206d with a base 208d. Accordingly, when worn by a user, a first strut (e.g., strut 300) may connect between female housing 206a and female housing 206d and a second strut may connect between female housing 206b and female housing 206c (see e.g., FIG. 8).

The housings 206 may be placed on the buccal side of the upper and lower alignment trays 204, 202. Further, the housings 206a, 206b of the upper alignment tray 204 may be positioned distal relative to the housings 206c, 206d of the lower alignment tray 202. In various embodiments, the housings 206a, 206b of the upper alignment tray 204 may be placed between or adjacent to one or more of the maxillary molars (e.g., first, second, or third molar) and the housings 206c, 206d of the lower alignment tray 202 may be placed between or adjacent to one or more of the mandibular molars (e.g., first molar) or premolars (e.g., first premolar or second premolar). For example, the housings 206a, 206b may be positioned between the second and third molars and the housings 206c, 206d may be positioned adjacent to the first molars. In the illustrated embodiment, the housings 206a, 206b are positioned adjacent to the second molars and the housings 206c, 206d are positioned between the second premolars and the first premolars.

The area of attachment of the female housings 206 will have a relatively thicker base 208 to prevent (at least in part) the female housing 206 from detaching from the tray when progressively changing the struts 300 or due to protrusion of the lower jaw. Further, the base 208 of the female housing 206 may be customized based on the dentition of a user to ensure robust attachment that aligns the housings 206 for receipt of struts (e.g., strut 300) while maintaining minimal discomfort to the user. Accordingly, in conjunction with ensuring robust attachment of the housings 206 and proper alignment, the protrusion of the housings 206 away from the buccal surface of the teeth may be minimized to maintain a comfortable fit when worn by the user. When housings 206 (e.g., housing 206c, 206d) are positioned between teeth, the bases 208 (e.g., bases 208c, 208d) may have a wedge shape to properly align the housings for receipt of struts. Similarly, when housings 206 (e.g., housings 206a, 206b) are positioned adjacent to teeth, the bases 208 (e.g., bases 208a, 208b) may have a concave shape to properly align the housings for receipt of struts. As described in more detail below, dental scans and/or impressions may be utilized in conjunction with manufacturing techniques (e.g., 3D printing) to accommodate the dentition of a user and achieve the proper positioning of the housings 206 with a comfortable fit. In various embodiments, the upper and lower alignment trays 204, 202 may be worn by a user without the struts installed, such as when the user is awake. Accordingly, the shape of the housings 206 may be configured to promote a comfortable fit with minimal user discomfort whether the struts are installed or not. For example, edges and corners of the housings may be rounded.

With reference to FIG. 8, an exemplary (representative) configuration of an oral appliance according to this disclosure is a combined oral appliance and orthodontic aligner 200 (“aligning appliance”) generally shown in partially exploded view. While the upper and lower trays (204, 202) are shown as pieces without an impression of dentition or other conformance, it is understood that FIG. 8 shows the general configuration of the oral appliance according to this disclosure, including the oral aligning appliance in which the upper alignment tray 204 and a lower alignment tray 202 may be used with struts 300 (i.e., progressive detachable arms) in the aligning appliance 200.

In an aspect, an exemplary method for forming and/or using the custom device, once the impression of the dentition is made, the patient will be furnished with a series of pre-determined custom sleep appliance trays (upper and lower arch), where the patient will wear each aligner/tray for a period of time—e.g., approximately 10 days—before advancing to the next tray set. All along, the patient's lower jaw is protruded, based on their therapeutic forward position, as dictated by their sleep healthcare professional. The protrusion of the lower jaw is done by exchanging the removeable struts 300 which are available in 0.5 mm measurements. Specifically, each strut 300 may be disconnected at both ends (302, 304 (FIG. 6 and FIG. 7)) from the female housing 206 to which it is attached (discussed further below) on each of the upper alignment tray 204 and the lower alignment tray 202. Progressively correcting the dentition alignment while treating OSA eliminates any unnecessary gap or delay in OSA treatment that may be lifesaving. Patients who do desire to correct their dentition alignment need not delay the initiation of OSA treatment because both treatments are combined in one device.

The aligning appliance 200 according to this disclosure may be worn without the struts 300 attached during non-asleep hours. The female housing 206 on either side of the upper alignment tray 204 and lower alignment tray 202 may have a rounded geometry to reduce or eliminate discomfort when wearing the aligners during non-sleep hours.

FIG. 6 and FIG. 7 show an exemplary strut 300 in isolation. FIG. 6 and FIG. 7 show the same strut 300 from respective top, left perspective and front views. It will be appreciated that the respective struts 300 on opposite sides of a tray will be configured as mirror images of each other.

The various parts of the aligning appliance 200 may generally be made from materials that are suitable for use in an oral cavity, including, for example, stainless steel or any other surgical grade metal alloy, or any biocompatible, non-metallic material (including BPA free material), such as polymeric materials (i.e., polymers). For example, the trays may be made of a polymeric material (i.e., polymer) having a shore hardness of from about 60 to about 70, such as polypropylene. The parts of the struts 300 may likewise be made of a polymeric material, for example, polycarbonate or polyester.

The various parts of the aligning appliance 200 may likewise be made in any suitable manner, for example, using injection molding, 3D printing, or any other suitable technique known in the art and consistent with this disclosure. In one aspect, the various parts of each alignment tray 202, 204 and strut 300 may be made from a moldable material, such as a polymeric material (e.g., a polymer), so that, e.g., each tray may be integrally formed with female housings at a desired position and the body 301 and projections 303, 305 of each strut 300 may be integrally formed to a desired length and configuration. The integral components that may fit together mechanically without fasteners or other components eliminates the need for screws, brackets, or other parts that could loosen and/or may be inadvertently lost or swallowed if not handled with extreme care. The interchangeability of progressive components also allows routine adjustment for either sleep apnea treatment, alignment, or both.

As is understood by those of skill in the art, orthodontic aligners are fabricated to accommodate the patient's teeth. The aligners are positioned on the teeth and held in place by specifically designed buttons or protrusive elements (not shown) attached to the patient's teeth. The aligners are changed periodically (e.g., every two weeks). As the aligners are progressively changed, the teeth move into alignment. To use such aligners with the aligning appliance 200 discussed herein, an orthodontic aligner is fabricated, e.g., typically using a vacuum forming process. According to an exemplary embodiment of the present disclosure, a method for forming and using the aligning appliance 200 includes forming the orthodontic aligners (i.e., upper and lower alignment trays 204, 202) and removably assembling the trays to the struts 300 as discussed further below. Upon completion of the orthodontic treatment with specific tray(s) and/or struts, the existing tray(s) and struts may be disassembled and a new combination of one or both trays and a new strut may be obtained and assembled in the same fashion. This process may be performed by a dentist, for example, every two weeks, as the aligners are replaced with new ones. This technique may be used, for example, for smile line correction for patients who desire an improvement in the alignment of their teeth, or to restore masticatory function while they undergo treatment with their oral appliance.

The exemplary strut 300—without limitation—may have a unitary construction including a unitary body 301. The unitary body 301 may be formed from a polymeric material. According to an aspect, the unitary body 301 includes an anterior end 302 and a posterior end 304. Additionally, the unitary body 301 may include a bend between 80 and 100 degrees, such as 90 degrees. The portion of the unitary body 301 between the bend and the anterior end 302 may be referred to as the anterior section and the portion of the unitary body between the bend and the posterior end 304 may be referred to as the posterior section. In various embodiments, a length of the posterior section may be less than a length of the anterior section. Further the inner and/or outer corners of the bend may be rounded to promote a comfortable fit for the user. The unitary body 301 may have a generally uniform thickness along its entire length (for example, between the anterior end 302 and the posterior end 304). According to an aspect, the length of the struts 300, as measured between the anterior end 302 and the posterior end 304, may be between about 20 to 30 millimeters (mm). According to an aspect, the length is about 25 mm. According to yet another aspect, the length is 25.3 mm. In some embodiments, the length of the anterior section of the unitary body may be between 15 and 30 mm and the length of the posterior section of the unitary body may be between 5 and 20 mm. In one embodiment, the length of the posterior section may be between one fifth and three fifths the length of the anterior section. For example, the length of the posterior section may be one half the length of the anterior section.

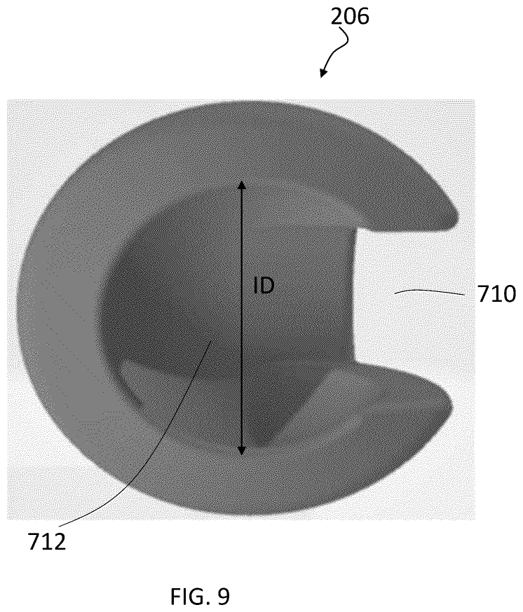

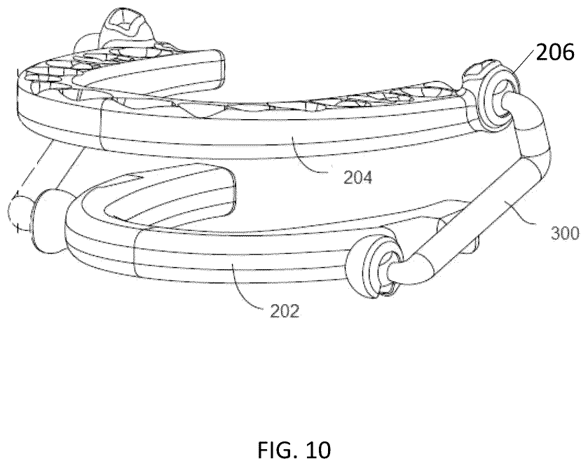

A posterior projection 305 is connected to the posterior end 304. The posterior projection 305 may be integrally and monolithically formed with the unitary body 301. In some embodiments, the entire strut 300 may be integrally and monolithically formed. According to an aspect, the posterior projection 305 has a maximum outer diameter (MOD), which may enable the posterior projection 305 to be secured or retained in the female housing 206. With continuing reference to FIGS. 6 and 7 and additional reference to FIG. 9 and FIG. 10, when the posterior projection 305 is secured in the female housing 206, it may be rotated up to a full 360-degrees and allows the upper alignment tray 204 and the lower alignment tray 202 to move from a first position in which the trays are adjacent as shown in FIGS. 1 and 8 to a second position in which the trays are separated as shown in FIG. 10.

An anterior projection 303 is connected to the anterior end 302 and is configured and functions as described for the posterior projection 305. For brevity, those aspects are not repeated.

With reference again to FIGS. 6 and 7, the anterior projection 303 and posterior projection 305 each include a pair of opposed planar surfaces 318a, 318b, such that the anterior projection 303 and the posterior projection 305 have a shape of a sphere sliced by two parallel planes, i.e., shaped as a spherical segment. The anterior projection 303 and posterior projection 305 may be connected respectively to the anterior end 302 and posterior end 304 at a tapered section 320. For example, the anterior projection 303 may be connected to the anterior end 302 at a first tapered section and the posterior projection 305 may be connected to the posterior end 304 at a second tapered section. The tapered section 320 may improve user comfort, such as by reducing pinch points between the strut and the alignment tray. In some embodiments, the anterior projection 303 and posterior projection 305 may be removably connected respectively to the anterior end 302 and posterior end 304.

According to an aspect, the unitary body 301, the posterior projection 305 and the anterior projection 303 are integrally formed. For example, the strut 300 may be a 3D printed structure. The strut 300 may be 3D printed from a polymeric material.

With reference to FIGS. 8 and 9, the lower alignment tray 202 and the upper alignment tray 204 respectively include two female housings 206 on opposite sides. The female housings on the lower alignment tray 202 are further forward than those on the upper alignment tray 204, to create an offset (or protrusive distance) prescribed by a dentist. In other words, the aligning appliance 300 is configured to position the bottom jaw of a user in a direction that is forward from the natural position of the user's jaw to aid in the treatment of sleep apnea in the user. The best jaw position for the user may be determined by a dentist and prescribed to a lab that is tasked with fabricating the oral appliance 300.

The female housings 206 extend outwardly from opposed sides of the upper alignment tray 204 and the lower alignment tray 202. The female housings 206 function as bearings that enable the struts 300 to freely move while the anterior projections 303 and the posterior projections 305 are secured in their respective female housing 206. The pair of female housings 206 on the upper alignment tray 204 may be positioned at a defined or constant location on the upper alignment tray 204, serving as a reference point for any desired protrusive distance changes that may need to be made to the lower alignment tray 202. Similarly, the length of each strut 300 may also be constant. The female housings 206 each comprise a round opening 712 having an inner diameter ID.

It is contemplated that the alignment trays 202, 204 will derive their geometry from a three-dimensional (3D) scan of the user's upper and lower teeth. A lab technician, for example, incorporates the female housings 206 into a computer aided design (CAD) model. The technician will then output a file for 3D printing in one of several methods. As described hereinabove, the female housings 206 on the upper alignment tray 204 may function as a reference plane from which to measure the positions of the female housings 206 on the lower tray 202.

The female housings 206 on the lower alignment tray 202 are positioned anteriorly to the female housings 206 of the upper alignment tray 204. According to an aspect, adjustments made to the relative positions of the female housings 206 on the lower alignment tray 202 impact the protrusive distance of the user's jaw. This means that a dentist can prescribed a desired protrusive distance and only the lower alignment tray 202 will require modification or replacement according to the user's needs.

Each female housing 206 according to the exemplary embodiments is configured for receiving a projection 303, 305 a corresponding strut 300. The female housing 206 may include a slit, slot, or gap 710 that helps to facilitate entry of the projection into the female housing 206. According to an aspect, the projections 303, 305 are removably coupled to corresponding female housings 206 by placement of the projections 303, 305 into the female housing 206. The slot 710 may be of a predetermined (or constant) width and may be configured to allow the projections 303, 305 to be connected to the female housing 206. The geometry of the projections 303, 305 and the alignment trays 202, 204 may be designed so that after the assembly of the strut 300 with the alignment trays 202, 204, while the aligning appliance 200 is in the user's mouth, the struts 300 will not be dislodged or disengaged from the alignment trays 202, 204. If it is determined that a different offset for the particular user is necessary, the lower alignment tray 202 with the revised offset may be the only tray that needs to be fabricated. However, any or all of the upper alignment tray 204, the struts 300, and the lower alignment tray 202 may be refabricated or substituted with different dimension(s) or configuration(s) for affecting the offset and aligning functions or another reason such as due to wear-and-tear.

In an aspect of the exemplary embodiments, each of the female housings 206 on the aligning appliance 200 has substantially the same configuration and interaction with a corresponding projection 303, 305 of the strut 300. Similarly, each of the struts 300 has substantially the same configuration including for each of the projections 303, 305. The opening 712 of the female housing 206 and the respective projections 303, 305 are shaped and dimensioned to be “keyed” to one another. That is, the projections 303, 305 can only be inserted into respective female housings 206 when the projections 303, 305 are properly aligned with (i.e., in “shape alignment” with) the gap 710 of the respective female housing 206. Specifically, in the illustrated embodiment, each projection 303, 305 can be inserted into the corresponding female housing 206 when the planar portions/surfaces 318a, 318b of the projection 303, 305 are aligned with the gap 710 in the female housing 206. Thus, it will be appreciated that the dimensions of the female housings 206 may be substantially the same as the dimensions of the projection 303, 305. In some embodiments, the dimensions of the projections 303, 305 may be slightly larger than certain respective dimensions of the female housings 206 to provide a “snap” fit when connecting the struts 300.

To attach each strut 300 to each of the alignment trays 202, 204 (after choosing a strut 300 of the appropriate length), the strut 300 must be oriented in a direction generally perpendicular to the plane of the tray such that, for example, the tray may be rotated vertically (e.g., at a 90-degree angle to socket female housing 206) to key the projections to the female housings via the gaps 710. Once the projections are removably connected with the corresponding female housings, the tray may then be reverted back to its horizontal position, so that the strut 300 and the tray are removably secured to one other. The procedure may be completed one tray at a time. As above, the projections 303, 305 may be dimensioned to snap into the female housings 206 if desired. If further adjustments are necessary, the above process may be reversed to detach the struts 300 and so on.

As discussed above, once the struts 300 have been set to the desired length and attached to the alignment trays 202, 204, the lower jaw is in a fixed position relative to the upper jaw. Thus, although the joints allow for some lateral and vertical movement of the lower alignment tray 202 relative to the upper alignment tray 204 to accommodate typical jaw movements, the lower jaw is prevented from moving in a retrusive (i.e., backward) direction away from the necessary treatment position. The lateral movement of the lower alignment tray 202 relative to the upper alignment tray 204 may be between 4 mm and 10 mm to either side of the midline of the face. The vertical movement of the lower alignment tray 202 relative to the upper alignment tray 204 may be between 2 mm and 8 mm for a habitual bite of a user. In one embodiment, the vertical movement of the lower alignment tray 202 relative to the upper alignment tray 204 may facilitate vertical movement between the biting surfaces of the alignment trays between 0 mm and 6 mm. In some embodiments, the thickness of the trays may be between 0.5 mm and 2 mm, such as 1 mm.

The struts 300 are configured for positioning the upper alignment tray 204 and the lower alignment tray 202 in an offset configuration so that the lower jaw of the user is urged forward relative to the upper teeth of the user. In an aspect, the aligning appliance 200 may be configured such that adjusting the position of the female housings 206 anteriorly or posteriorly on the lower alignment tray 202 impacts the range of protrusion that is obtained for a user's mandible. In another aspect, the struts 300 are replaceable with struts of different length. The aligning appliance is configured overall to provide the sleep apnea treatment and aligning function concurrently.

In other aspects of various embodiments, the geometry of the female housings 206 may include a canter angle of about 6-degrees to the horizontal plane to help facilitate increased motion of the struts 300, while also maintaining engagement of the projections 303, 305 with the female housings 206. It is contemplated that other canter angles other than 6-degrees may be prescribed depending on the profile curve of the patient's jaw. Some patients have a wider, more elliptical shaped curvature while others have a sharper V-shaped curvature. For example, the canter angle may be between 1-degree and 8-degrees or between 4-degrees and 8-degrees.

The positioning of the female housings 206 on the respectively alignment trays 202, 204 may be based on a prescribed treatment plan of a dentist. The canter angle of the projections 303, 305 may be parallel to a corresponding canter angle of the corresponding female housings 206. This may ensure the maximum articulation of the lower alignment tray 202 relative to the upper alignment tray 204, for example when the user's bottom jaw translates laterally, is up to about 8 mm. It is contemplated that since the struts 300 can articulate in both the upper and lower alignment trays 202, 204, there is no binding or restricted movement of the struts 300, and therefore reduce the likelihood that undue forces on the struts 300 may potentially result in failure of the struts 300. Failure may be defined by the struts 300 becoming permanently deformed, fractured or broken due to forces applied on the struts 300.

According to an aspect, the positions of the female housings 206 may be adjusted by incremental changes, such as based on the jaw anatomy of a user. For example, the incremental changes may be in 0.5 mm increments. To be sure, changes may be in increments of fraction of a millimeter or more, based on the dentist's discretion. According to an aspect, the incremental changes may facilitate an offset of up to about 10 mm. The incremental changes may facilitate an offset more than 10 mm or more than 15 mm, all depending on the dentist's prescribed treatment of the patient.

It is contemplated that one or more sensors (not shown) may be positioned on or housed within at least one of the upper alignment tray 204 and the lower alignment tray 202. These sensors may be used to collect physiological data, and in some instances, monitoring and diagnose obstructive sleep apnea (OSA). In some instances, the sensors may also be able to provide a therapeutic to treat the OSA. It is contemplated that any captured physiological data can be used to monitor the efficacy of the treatment prescribed, as well as serves as a trigger for initiating therapy for OSA. Such sensors may include, for example, an accelerometer/gyroscope, an oral microphone, a piezo-pressure sensor, an oxygen sensor and a wireless electroencephalogram (EEG).

According to an aspect, a custom oral appliance may be fabricated by either digitally scanning the patient's jaw/dentition or taking conventional impression molds. A clear retainer type aligner may be fabricated according to known techniques. After scanning or using impression material to capture the dentition, the laboratory will fabricate custom oral appliance alignment trays (e.g., top and bottom trays) that may include a cured and/or adhered lining that can be inserted over the already fabricated progressive alignment trays.

The present disclosure, in various embodiments, configurations and aspects, includes components, methods, processes, systems and/or apparatus substantially developed as depicted and described herein, including various embodiments, sub-combinations, and subsets thereof. Those of skill in the art will understand how to make and use the present disclosure after understanding the present disclosure. The present disclosure, in various embodiments, configurations and aspects, includes providing devices and processes in the absence of items not depicted and/or described herein or in various embodiments, configurations, or aspects hereof, including in the absence of such items as may have been used in previous devices or processes, e.g., for improving performance, achieving ease and/or reducing cost of implementation.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, and “A, B, and/or C” means A alone, B alone, C alone, A and B together, A and C together, B and C together, or A, B and C together.

In this specification and the claims that follow, reference will be made to a number of terms that have the following meanings. The terms “a” (or “an”) and “the” refer to one or more of that entity, thereby including plural referents unless the context clearly dictates otherwise. As such, the terms “a” (or “an”), “one or more”, and “at least one” can be used interchangeably herein. Furthermore, references to “one embodiment”, “some embodiments”, “an embodiment”, and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first”, “second”, “upper”, “lower”, etc. are used to identify one element from another, and unless otherwise specified are not meant to refer to a particular order or number of elements. All directional references (e.g., “upper”, “lower”, “upward”, “downward”, “left”, “right”, “leftward”, “rightward”, “top”, “bottom”, “above”, “below”, “vertical”, “horizontal”, “clockwise”, and “counterclockwise”) are used only for identification purposes to aid the reader's understanding of the various embodiments of the present invention, and do not create limitations, particularly as to the position, orientation, or use of the invention unless specifically set forth in the claims.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that variations in these ranges will suggest themselves to a practitioner having ordinary skill in the art and, where not already dedicated to the public, the appended claims should cover those variations.

The terms “determine”, “calculate”, “compute” and variations thereof, as used herein, are used interchangeably and include any type of methodology, process, mathematical operation, or technique.

The foregoing discussion of the present disclosure has been presented for purposes of illustration and description. The foregoing is not intended to limit the present disclosure to the form or forms disclosed herein. In the foregoing Detailed Description for example, various features of the present disclosure are grouped together in one or more embodiments, configurations, or aspects for the purpose of streamlining the disclosure. The features of the embodiments, configurations, or aspects of the present disclosure may be combined in alternate embodiments, configurations, or aspects other than those discussed above. This method of disclosure is not to be interpreted as reflecting an intention that the present disclosure requires more features than are expressly recited in each claim. Rather, as the following claims reflect, the claimed features lie in less than all features of a single foregoing disclosed embodiment, configuration, or aspect. Thus, the following claims are hereby incorporated into this Detailed Description, with each claim standing on its own as a separate embodiment of the present disclosure.

Advances in science and technology may make equivalents and substitutions possible that are not now contemplated by reason of the imprecision of language; these variations should be covered by the appended claims. This written description uses examples to disclose the method, machine and computer-readable medium, including the best mode, and also to enable any person of ordinary skill in the art to practice these, including making and using any devices or systems and performing any incorporated methods. The patentable scope thereof is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

What is claimed is:

1. A strut for an oral aligning appliance, the strut comprising:
a unitary body formed from a polymeric material, the unitary body having an anterior end and a posterior end;
a posterior projection connected to the posterior end; and
an anterior projection connected the anterior end,
wherein the anterior projection and the posterior projection are each shaped as a spherical segment.

2. The strut of claim 1, wherein the posterior projection and the anterior projection are respectively configured for removably connecting to corresponding female housings of the oral aligning appliance.

3. The strut of claim 1, wherein the posterior projection is connected to the posterior end at a first tapered section, and the anterior projection is connected to the anterior end at a second tapered section.

4. The strut of claim 1, wherein the posterior projection and the anterior projection are integrally and monolithically formed with the unitary body.

5. The strut of claim 1, wherein the unitary body includes a bend between 80 and 100 degrees.

6. The strut of claim 5, wherein the bend is 90 degrees.

7. The strut of claim 5, wherein a length between the bend and the anterior end is greater than a length between the bend and the posterior end.

8. The strut of claim 5, wherein an outer corner of the bend is rounded.

9. An oral aligning appliance for concurrently treating sleep apnea and aligning dentition in a user, the oral aligning appliance comprising:
an upper alignment tray configured for receiving upper teeth of the user;
a first female housing positioned on a first side of the upper alignment tray and a second female housing positioned on an opposite side of the upper alignment tray;
a lower alignment tray configured for receiving lower teeth of the user;
a first female housing positioned on a first side of the lower alignment tray and a second female housing positioned on an opposite side of the lower alignment tray; and
a pair of struts pivotably joined to each of the upper alignment tray and the lower alignment tray, wherein each strut includes a body, a posterior projection, and an anterior projection, wherein each of the posterior projections and the anterior projections is removably connected to a corresponding female housing, and
each of the anterior projections and the posterior projections is shaped as spherical segment.

10. The oral aligning appliance of claim 9, wherein each female housing has the same dimensions and each of the anterior projections and the posterior projections have the same dimensions.

11. The oral aligning appliance of claim 9, wherein each of the upper and lower alignment trays is replaceable as part of the aligning the dentition of the user.

12. The oral aligning appliance of claim 9, wherein the first female housing positioned on the first side of the upper alignment tray includes a first base with a first wedge shape and the second female housing positioned on the opposite side of the upper alignment tray includes a second base with a second wedge shape.

13. The oral aligning appliance of claim 9, wherein the first female housing positioned on the first side of the lower alignment tray includes a first base with a first concave shape and the second female housing positioned on the opposite side of the upper alignment tray includes a second base with a second concave shape.

14. The oral aligning appliance of claim 9, wherein the first female housing positioned on the first side of the upper alignment tray includes a first base with a first concave shape and the second female housing positioned on the opposite side of the upper alignment tray includes a second base with a second concave shape.

15. The oral aligning appliance of claim 9, wherein the first female housing positioned on the first side of the lower alignment tray includes a first base with a first wedge shape and the second female housing positioned on the opposite side of the upper alignment tray includes a second base with a second wedge shape.

16. A method for forming and/or using an oral aligning appliance including a strut according to any of the previous claims, comprising:
making an impression of a user's dentition;
fabricating a series of one or both of upper and lower alignment trays for treating sleep apnea while progressively aligning the dentition of the user;
removably connecting a first strut to each of a first upper alignment tray and a first lower alignment tray;
removing the first strut from one or both of the first upper alignment tray and the first lower alignment tray;
replacing at least one of the first strut, the first upper alignment tray, and the first lower alignment tray with a corresponding at least one of a second strut, a second upper alignment tray, and a second lower alignment tray, and reassembling the oral aligning appliance with the at least one of the second strut, second upper alignment tray, and second lower alignment tray, as part of the progressively aligning the dentition or treating sleep apnea.

17. The method of claim 16, wherein the removing and replacing steps occur progressively at predetermined periods of time.

18. The method of claim 16, wherein the patient's lower jaw is protruded based on their therapeutic forward position and the protrusion of the lower jaw is done by exchanging the first strut for a second strut with a length approximately 0.5 mm different than a length of the first strut.

19. The method of claim 16, wherein the first strut is removed by disconnecting one or both of the anterior or posterior projections from corresponding female housings on the lower alignment tray or the upper alignment tray, and the oral aligning appliance is reassembled by removably connecting the first strut or the second strut to the corresponding female housings on the second lower alignment tray or the second upper alignment tray.

20. The method of claim 19, wherein each of the female housings has the same dimensions, the second strut has an anterior projection and a posterior projection, and each of the anterior projections and the posterior projections on each of the first strut and the second strut has the same dimensions as the others.
